(19) United States
(12) Patent Application Publication
(10) Pub. No.: US 2024/0327500 A1
(43) Pub. Date: Oct. 3, 2024

(54) ANTI-HBV ANTIBODIES AND USES THEREOF

(71) Applicants: National University of Singapore, Singapore (SG); National University Hospital (Singapore) Pte Ltd, Singapore (SG); Agency for Science, Technology and Research, Singapore (SG)

(72) Inventors: Paul Anthony Macary, Singapore (SG); Saket Jhajharia, Singapore (SG); Seng Lim, Singapore (SG); Qingfeng Chen, Singapore (SG); Fritz Lai, Singapore (SG)

(73) Assignees: National University of Singapore, Singapore (SG); National University Hospital (Singapore) Pte Ltd, Singapore (SG); Agency for Science, Technology and Research, SG (SG)

(21) Appl. No.: 18/579,482

(22) PCT Filed: Jul. 6, 2022
(86) PCT No.: PCT/SG2022/050468

§ 371 Date: Jan. 15, 2024

(30) Foreign Application Priority Data
Jul. 15, 2021 (SG) 10202107784Y

(51) Int. Cl. C07K 16/08; A61K 39/00; A61P 31/20; G01N 33/58; G01N 33/68

(52) U.S. Cl. CPC C07K 16/082; A61P 31/20; G01N 33/58; G01N 33/6854; A61K 2039/505; C07K 2317/14; C07K 2317/32; C07K 2317/565; C07K 2317/76; G01N 2333/02

(57) ABSTRACT

The present invention relates to isolated and engineered human antibodies that specifically bind to at least one conformational (non-linear) epitope of HBsAg and neutralize hepatitis B virus infection, methods for producing the same, and uses thereof in treating human HBV infection.

### FIELD OF THE INVENTION

The present invention relates to isolated and engineered human antibodies that specifically bind to at least one conformational (non-linear) epitope of HBsAg and neutralizes hepatitis B virus infection, method for producing the same, and uses thereof in treating human HBV infection.

### BACKGROUND OF THE INVENTION

Hepatitis B Virus (HBV) infection accounted for 1.34 million deaths in 2015 and WHO has estimated that 257 million individuals are chronically infected worldwide [WHO. Global Hepatitis Report 2017]. HBV causes either an acute or chronic infection, with a high possibility of the latter resulting in liver cirrhosis or hepatocellular carcinoma (HCC) which are the leading causes of mortality [WHO. Global Hepatitis Report 2017; Trepo, C., et al., The Lancet 384:2053-2063 (2014)]. Current therapeutic options for HBV have an efficacy of 1-2% annually in achieving a functional cure [Lok, A. S., et al., Hepatology 66:1296-1313 (2017)]. The pivotal importance of an effective B-cell/antibody response for achieving a functional cure was shown in patients receiving B-cell depleting therapies—this results in the reactivation of HBV in resolved individuals [Palanichamy, A. et al., J Immunol 193:580-586 (2014).; Tsutsumi, Y. et al., World J Hepatol 7:2344-2351 (2015); Chen, K. L. et al., Chin J Cancer 34:225-234 (2015)]. In terms of the dominant IgG subclasses produced in convalescent individuals, previous studies have reported that an IgG1 response is most dominant followed by IgG3 and IgG4 responses [Tsai, T. H. et al., Viral Immunol 19:277-284 (2006); Gregorek, H. et al., J Infect Dis 181:2059-2062 (2000)]. Anti-HBs IgG4 antibodies are more abundant in chronically infected patients and mostly confined to antibody-HBsAg immune complexes that are not efficiently cleared from circulation due to weak FcyR binding [Rath, S. & Devey, M. E. Clin Exp Immunol 72:164-167 (1988)].

There are three membrane-embedded surface proteins (L, M, and S) on HBV with a common S antigenic region [Julithe, R., et al., J Virol 88:9049-9059 (2014)]. The S region contains the ‘a’ determinant spanning from residues 99 to 160 and is thought to be the major anti-HBs binding domain [Alavian, S. M., et al., J Clin Virol 57:201-208 (2013)]. Antibodies targeting the ‘a’ determinant have been reported to be potent neutralizers, making them a high-value modality for testing as a prophylactic or therapeutic reagent [Walsh, R. et al., Liver Int 39:2066-2076 (2019)].

Several human or humanized antibodies against HBsAg have been isolated by other groups using a variety of discovery methods including: monoclonal hybridoma technology [Kucinskaite-Kodze, I. et al., Virus Res 211:209-221 (2016); Zhang, T. Y. et al., Gut 65:658-671 (2016)]; Phage-FAB display [Li, D. et al., Elife 6: (2017); Kim, S. H. & Park, S. Y. Hybrid Hybridomics 21:385-392 (2002); Wang, W. et al., MAbs 8:468-477 (2016)]; humanized mice [Eren, R. et al., Immunology 93:154-161 (1998)]; and human B cell cultures [Cerino, A., et al., PLoS One 10: e0125704 (2015); Heijtink, R. A. et al., J Med Virol 66:304-311 (2002)]. These antibodies were isolated by screening against recombinant viral proteins.

Hepatitis B Immunoglobulin (HBIG) is currently the prophylactic agent of choice employed in at-risk individuals [Crespo, G., et al., Gastroenterology 142:1373-1383 e1371 (2012); Both, L. et al., Vaccine 31:1553-1559 (2013)]. However, HBIG is plagued by limited availability, batch variation, and low specific activity [Crespo, G., et al., Gastroenterology 142:1373-1383 e1371 (2012); Li, D. et al., Elife 6: (2017)]. The lack of efficacious therapeutic and prophylactic options translates into a clear requirement for new modalities to improve the clinical management of HBV.

Therefore, there is a need for improved HBV antibodies for clinical use.

### SUMMARY OF THE INVENTION

The present invention relates to the development of an anti-HBsAg-S fully human monoclonal antibody termed mAb006-11. mAb006-11 was isolated from an HBV convalescent patient by screening directly against HBV (Genotype D). We show that mAb006-11 binds to a conformational epitope on HBsAg found on all four major genotypes and has potent neutralizing activity. Next, we engineered recombinant human IgG subclass variants of mAb006-11 to test if the subclass can influence the binding and neutralizing activity for HBV. Although mAb006-11 was isolated from an IgG1 subclass, the IgG4 subclass showed significantly better neutralizing potential in in-vitro assays. Finally, mAb006-11-IgG1 is reported to show superior prophylactic and therapeutic utility in an in-vivo setting when compared to HBIG. As a post-infection anti-viral agent, mAb006-11 significantly reduced HBV DNA levels and circulating HBsAg. These findings indicate that mAb006-11 represents a potential prophylactic or therapeutic modality for medical intervention in HBV.

In a first aspect, the present invention provides an isolated neutralizing antibody or a fragment thereof that specifically binds to at least one conformational (non-linear) epitope of HBsAg and neutralizes infection with hepatitis B virus, wherein the antibody comprises variable light chain CDRL1, CDRL2 and CDRL3 amino acid sequences comprising the amino acid sequence set forth in SEQ ID NO 3, SEQ ID NO 4, and SEQ ID NO 5, and variable heavy chain CDRH1, CDRH2, and CDRH3 amino acid sequences comprising the amino acid sequence set forth in SEQ ID NO 6, SEQ ID NO 7, and SEQ ID NO: 8.

Antibody fragments that contain the idiotype of the antibody molecule can be generated by known techniques. For example, such can be produced by pepsin digestion of the antibody molecule; the Fab fragments can be generated by reducing the disulfide bridges of the F(ab′) 2 fragment, and the Fab fragments which can be generated by treating the antibody molecule with papain and a reducing agent. Such antibody fragments can be generated from any of the antibodies of the invention.

In some embodiments, the antibody comprises at least one variable light chain and at least one variable heavy chain, wherein the variable light chain amino acid sequence comprises the amino acid sequence set forth in SEQ ID NO: 1, and the variable heavy chain amino acid sequence comprises the amino acid sequence set forth in SEQ ID NO: 2.

In some embodiments, the antibody has a constant region selected from the group comprising IgA, IgG1, IgG2, IgG3 and IgG4 subclass scaffolds, preferably an IgG4 subclass scaffold.

In some embodiments, the antibody is a fully-human antibody.

In some embodiments, the hepatitis B virus is selected from the group comprising genotype A (adw2), genotype B (adw), genotype C (adr) and genotype D (ayw).

In a second aspect, the invention provides a pharmaceutical composition comprising the isolated neutralizing antibody or fragment thereof, of any aspect of the invention, and a pharmaceutically acceptable carrier.

In a third aspect, the invention provides an isolated neutralizing antibody, fragment thereof, or pharmaceutical composition comprising same according to any aspect of the invention for use in the prophylaxis or treatment of HBV infection and/or at least one HBV-linked disease.

In some embodiments, the prophylaxis or treatment is for prophylaxis of perinatal HBV transmission, treatment of HBV positive mothers during the third trimester of pregnancy, prophylaxis of HBV recurrence in liver transplant recipients or HBV post-exposure prophylaxis in vaccine non-responders exposed to HBV or HBsAg-positive materials.

In a fourth aspect, the invention provides a use of at least one antibody or fragment thereof defined according to any aspect of the invention for the preparation of a medicament for prophylaxis or treatment of HBV infection and/or at least one HBV-linked disease.

In a preferred embodiment, the medicament comprises the fully-human antibody or a fragment thereof of the invention.

In some embodiments, the prophylaxis or treatment is for prophylaxis of perinatal HBV transmission, treatment of HBV positive mothers during the third trimester of pregnancy, prophylaxis of HBV recurrence in liver transplant recipients or HBV post-exposure prophylaxis in vaccine non-responders exposed to HBV or HBsAg-positive materials.

In a fifth aspect, the invention provides a method of prophylaxis or treatment of HBV-infection and/or at least one HBV-linked disease, the method comprising administering to a subject an antibody or fragment thereof according to any aspect of the invention.

In some embodiments, the prophylaxis or treatment is for prophylaxis of perinatal HBV transmission, treatment of HBV positive mothers during the third trimester of pregnancy, prophylaxis of HBV recurrence in liver transplant recipients, HBV post-exposure prophylaxis in vaccine non-responders exposed to HBV or HBsAg-positive materials or treatment of chronically infected individuals.

Effective dosages and schedules for administering the antibodies, fragments thereof and compositions of the invention may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, [e.g., Ferrone, S., & Dierich, M. P., Handbook of monoclonal antibodies: Applications in biology and medicine. Noges Publications, Park Ridge, N.J. (1985) ch. 22 and pp. 303-357; Smith, T. W., et al., In: Antibodies in Human Diagnosis and Therapy. Haber, E., Krause, R. M. (eds.) New York: Raven Press (1977) pp 365-389].

A typical daily dosage of the antibody used alone might range from about 1 μg/kg to up to 100 mg/kg of body weight or more per day, depending on the factors mentioned above.

In a sixth aspect, the invention provides an isolated nucleic acid molecule encoding
(a) at least one variable light chain of the neutralizing antibody of the invention; and
(b) at least one variable heavy chain of the neutralizing antibody of the invention.

In some embodiments, the nucleic acid molecule encodes;
(a) at least one variable light chain comprising the amino acid sequence set forth in SEQ ID NO: 1; and
(b) at least one variable heavy chain comprising the amino acid sequence set forth in SEQ ID NO: 2.

In some embodiments, the at least one nucleic acid sequence in (a) has at least 80%, at least 85%, at least 90%, at least 95% or 100% sequence identity to the nucleic acid sequence set forth in SEQ ID NO: 9; and
the at least one nucleic acid sequence in (b) has at least 80%, at least 85%, at least 90%, at least 95% or 100% sequence identity to the nucleic acid sequence set forth in SEQ ID NO: 10, due to redundancy of the genetic code.

The isolated nucleic acid molecule according to the invention may be cloned into an expression vector, which may in turn be transformed into a host cell for the production of an antibody according to any aspect of the present invention.

In an eighth aspect, the invention provides an expression vector comprising at least one isolated nucleic acid molecule defined above.

In a ninth aspect, the invention provides a host cell comprising the expression vector defined above.

In particular, the host cell may be human embryonic kidney (HEK) 293 cells, Chinese hamster ovary (CHO) cells or recombinant plant cells. Methods of using plant cells to produce human therapeutic antibodies are described in Qiu et al., [Nature 514 (7520): 47-53 (2014), incorporated herein by reference].

In a tenth aspect, the invention provides a kit comprising at least one neutralizing antibody, fragment thereof or composition as defined above.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended statements.

### BRIEF DESCRIPTION OF THE FIGURES

FIGS. 1A-E show mAb006-11 discovery and characterization. A. Schematic representation of antibody discovery methodology. B. Flow cytometric data for the selection of memory B cells for high-throughput antibody discovery C. mAb006-11 binding specificity was assessed at 5 μg/ml against live virus, recombinant HBsAg-S and recombinant PreS1/2 protein. D. The neutralizing potential of mAb006-11 & HBIG was tested via an in-vitro assay utilizing HBV (genotype D) and the HepG2-hNTCP cell line. E. The binding potential of mAb006-11 to the four most common HBV genotypes was tested via an ELISA assay, statistical analysis was performed using the T-test, *P<0.05 (Table 2). All data were expressed as mean±SEM, N=4 independent experiments.

FIGS. 2A-E show the epitope binding and influence of IgG subclasses on binding and in-vitro neutralization potential. A. Binding of mAb006-11 to HBsAg virus-like-particles interferes with/reduces detection by Bio-Plex mAbs specific for the loop 2 regions (antibodies 8 and 17). B. Predicted HBsAg structural data (I-Tasser) with highlighted loop 2 region indicating the broad binding region of mAb006-11. C. Heavy and light chains of mAb006-11 for the four principal IgG subclasses were resolved on polyacrylamide gel under reducing and non-reducing conditions. D. Binding specificity of four principal IgG subclasses of mAb006-11 at 5 μg/ml to recombinant HBsAg was tested by ELISA (mean±SEM, N=4 independent experiments) E. Affinity and Bₘₐₓ measurements of mAb006-11 subclass by QCM (mean±SEM, N=3 independent experiments). The dissociation equilibrium constant (K_{D}) and Bₘₐₓ for each subclass are indicated at the top right-hand corner.

FIGS. 3A-C show the development and optimization of capture ELISA for live virus screening assay. A. A schematic representation of the assay. B. Testing of two commercial mouse antibodies for determining the capture antibody reagent. Antibodies were compared by immunoprecipitation to determine which antibody capture virus more effective based on HBV genome copy number comparison. C. Optimization of the capture ELISA assay, with a primary antibody (human) at varying concentrations and a secondary goat anti-human antibody.

FIGS. 4A-D show the discovery and binding characterization of mAb006-11. A. B cells gating strategy and flow sorting for antibody discovery B. Isolation of mAb006-11 was done by performing a double screen against live virus and recombinant HBsAg. The wells within the box were picked for sequence recovery. C. Immunoblot detection of mAb006-11-IgG1 binding recombinant HBsAg under reducing (SDS+2-Betametarcarponal) and non-reducing (SDS only) conditions. Commercial goat anti-HBs (polyclonal) was used as a positive control. The protein ladder is labeled. D. No binding was detected by mAb006-11 to linear peptides of HBsAg loop 1 and loop 2 regions. This indicates that mAb006-11 is binding to a conformational epitope.

FIGS. 5A-D show the comparison of the four IgG subclasses of mAb006-11 and HBIG based on neutralizing potential A. Schematic representation of in-vitro neutralization assay. Neutralization data comparing the four principal subclasses of mAb006-11 and HBIG based on B. secreted HBsAg C. secreted HBeAg D. Representative flow data for intracellular HBcAg quantification used to plot the HBcAg neutralization curve.

FIGS. 6A-B show the gating strategy and measurement of intracellular HBcAg by flow cytometry. A. Gating strategy used for quantification of intracellular HBcAg on Day 7 post-infection. HepG2-hNTCP infected cells were quantified by staining for BV510-(live/dead stain) and AF647+ (Goat-anti Mouse secondary antibody binding to mouse anti-HBcAg). B. Flow plots for the various concentrations of the four IgG subclasses of mAb006-11 and HBIG, no virus, and virus only wells (representative plot from one experiment is shown). * HBIG concentration was 10× higher (3000 μg/ml, 300 μg/ml, 30 g/ml, 3 μg/ml, 0.3 μg/ml, 0.03 μg/ml)

FIGS. 7A-J show in-vivo characterization of mAb006-11. A. Schematic representation of human liver chimeric mice generation. B. Production of hALB in all mice was measured at ˜5 mg/ml. C. Schematic diagram indicative of antibody injection, virus inoculation, and serum sampling for prophylactic treatment. Weekly quantification of D. HBV DNA (Statistically analysis between mAb006-11 and Control mAb was performed using a t-test) and E. HBsAg production (Statistically analysis between mAb006-11 and Control mAb was performed using a F-test) in mice injected with Control mAb, HBIG, or mAb006-11 one day before HBV inoculation of up to 35 dpi. F. Histological analysis of mouse liver sections stained with hFAH, HBcAg, and H&E. G. The schematic diagram for therapeutic treatment indicative of antibody injection, virus inoculation, and serum sampling. Weekly quantification of H. HBV DNA (Statistically analysis between mAb006-11 and HBIG was performed using a F-test) and I. HBsAg production (Statistically analysis between mAb006-11 and HBIG was performed using a F-test) in mice injected with Control mAb, HBIG, or mAb006-11 at 42 dpi following the establishment of HBV infection. Further quantification was performed at 1-4 days post-antibody injection intervals for 15 days. J. Histological analysis of mouse liver sections stained with hFAH, HBcAg, and H&E. * P<0.05, ** P<0.005 *** P<0.0005

FIGS. 8A-E show the prophylactic validation of mAb006-11 in vivo. A. Production of hALB in all mice was measured at ˜7 mg/ml. Weekly B. body weight (g) changes and C. HBV DNA quantification in mice injected with Control mAb or mAb006-11 one day before HBV inoculation of up to 63 dpi. D. Measurement of HBsAg production in all mice at 63 dpi. E. Histological analysis of mouse kidney and spleen sections stained with H&E.

FIGS. 9A-D show the therapeutic validation of mAb006-11 in vivo. A. Production of hALB in all mice was measured at ˜4 mg/ml. B. Weekly quantification of HBV DNA in mice injected with Control mAb or mAb006-11 at 42 dpi following the establishment of HBV infection. Further quantification was performed at 1-3 days post-antibody injection intervals for 8 days. C. HBsAg production was measured at 42 dpi followed by 1-3 days post-antibody injection intervals for 8 days. D. Histological analysis of mouse kidney and spleen sections stained with H&E.

FIGS. 10A-B show a comparison of mAb006-11 and HBC34. A. The neutralization ability of mAb006-11 and HBC34 were compared via an in-vitro assay. As seen from the data above both the G1 and G4 versions of mAb006-11 were superior in neutralizing ability when compared to HBC34. B. When comparing the binding epitope of mAb006-11 and HBC34, it was noted that mAb006-11 does not bind to HBV S-antigen under reducing or non-reducing conditions, however this was not the case with HBC34 which is shown to have strong binding to S-antigen under non-reducing conditions and weak binding under reducing conditions.

### DETAILED DESCRIPTION OF THE INVENTION

Bibliographic references mentioned in the present specification are for convenience listed at the end of the examples. The whole content of such bibliographic references is herein incorporated by reference.

### Definitions

For convenience, certain terms employed in the specification, examples and appended claims are collected here.

As used herein, the term “antibody” refers to any immunoglobulin or intact molecule that bind to a specific epitope. Such antibodies include, but are not limited to monoclonal, chimeric, humanised and fully-human antibodies. The term “monoclonal antibody” may be referred to as “Mab”. The antibody includes fully human antibodies HDAC006-11, in IgG1 and IgG4 form. The heavy and light chain variable regions of antibody HDAC006-11 may be cloned into plasmids with the respective human IgG (IgG1, IgG2, IgG3, IgG4) constant regions to produce engineered HDAC006-11 antibodies in IgG1, IgG2, IgG3 or IgG4 form. The antibodies HDAC006-11, in IgG1 and IgG4 form, are capable of specifically binding to Hepatitis B Virus (HBV), including but not limited to a conformational epitope comprising at least one HBsAg protein of HBV.

The term “antibody fragment” as used herein refers to an incomplete or isolated portion of the full sequence of the antibody which retains the antigen binding function of the parent antibody. Examples of antibody fragments include single chain, single chain fragment variable (scFv), Fab, Fab′, F(ab′) 2, fragments and/or Fv portions; diabodies; linear antibodies; single-chain antibody molecules; and multi-specific antibodies formed from antibody fragments. Fragments of the HDAC006-11 antibodies are encompassed by the invention so long as they retain the desired affinity of the full-length antibody.

The term “antigen” as used herein, refers to a substance that prompts the generation of antibodies and can cause an immune response. It may be used interchangeably in the present invention with the term “immunogen”. In the strict sense, immunogens are those substances that elicit a response from the immune system, whereas antigens are defined as substances that bind to specific antibodies. An antigen or fragment thereof may be a molecule (i.e. an epitope) that makes contact with a particular antibody. When a protein or a fragment of a protein is used to immunize a host animal, numerous regions of the protein may induce the production of antibodies (i.e. elicit the immune response), which bind specifically to the antigen (given regions or three-dimensional structures on the protein). Non-limiting examples of an antigen is the HBsAg protein of HBV. The antigen may include but is not limited to a surface protein of HBV. In particular, the term “epitope” may refer to a consecutive sequence of from about 5 to about 13 amino acids which form an antibody binding site. The epitope in the form that binds to the antibodies or binding protein may be a denatured protein that is substantially devoid of tertiary structure. The epitope may be a conformational epitope that comprises non-consecutive elements from non-consecutive sequences. The epitope may also be a quaternary epitope which comprises non-consecutive elements from more than one protein or polypeptide which are assembled into a particle such as a virion or an assembly of some other kind.

A “conformational epitope” is herein defined as a sequence of subunits (usually, amino acids) comprising an antigen that comes in direct contact with the Variable Light and Heavy chains of an antibody. Whenever an antibody interacts with an undigested antigen, the surface amino acids that come in contact may not be continuous with each other if the protein is unwound. Such discontinuous amino acids that come together in three-dimensional conformation and interact with the antibody's paratope are called conformational epitopes. In contrast, if the antigen is digested, small segments called peptides are formed, which bind with major histocompatibility complex molecules, and then later with T cell receptors through amino acids that are continuous in a line. These are known as linear epitopes.

The term “comprising” is herein defined to be that where the various components, ingredients, or steps, can be conjointly employed in practicing the present invention. Accordingly, the term “comprising” encompasses the more restrictive terms “consisting essentially of” and “consisting of.”

The term “immunological binding characteristics” of an antibody or related binding protein, in all of its grammatical forms, refers to the specificity, affinity and cross-reactivity of the antibody or binding protein for its antigen.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

The term “neutralising antibody” is herein defined as an antibody that can neutralise the ability of that pathogen to initiate and/or perpetuate an infection in a host. The invention provides at least one neutralising human monoclonal antibody, wherein the antibody recognises an antigen from HBV.

The term “mutant” is herein defined as one which has at least one nucleotide sequence that varies from a reference sequence via substitution, deletion or addition of at least one nucleic acid, but encodes an amino acid sequence that retains the ability to recognize and bind the same conformational epitope on HBV as the un-mutated sequence encodes. In particular, the mutant may have at least 80%, at least 85%, at least 90%, at least 95% or 100% sequence identity to the reference sequences, due to the degeneracy of the genetic code, and still encode the amino acid sequences of the heavy and light chains of the antibody. The term ‘mutant’ also applies to an amino acid sequence that varies from at least one reference sequence via substitution, deletion or addition of at least one amino acid, but retains the ability to recognize and bind the same conformational epitope on HBV as the un-mutated sequence. In particular, the mutant may have at least 90%, or at least 95% sequence identity to the reference sequences.

The term “variant”, as used herein, refers to an amino acid sequence that is altered by one or more amino acids, but retains the ability to recognize and bind the same conformational epitope on HBV as the non-variant reference sequence. The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological or immunological activity may be found using computer programs well known in the art, for example, DNASTAR® software (DNASTAR, Inc. Madison, Wisconsin, USA).

The term “pharmaceutically acceptable” refers to a material that is not biologically or otherwise undesirable, i.e., the material may be administered to a subject, along with the antibody or active fragment thereof, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, P A 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

As used herein, the terms “specific binding” or “specifically binding” refer to the interaction between one or more proteins or peptides and an agonist, an antibody, or an antagonist. In particular, the binding is between an antigen and an antibody. The interaction is dependent upon the presence of a particular structure of the one or more proteins recognized by the binding molecule (i.e., the antigen or epitope). As described hereinbefore, the antigen or epitope may be comprised of more than a single peptide sequence from the same protein or different proteins which come together spatially to form a conformational antigen or epitope. For example, if an antibody is specific for epitope “A”, the presence of a polypeptide containing the epitope A or free unlabeled A, in a reaction containing free labeled A and the antibody, will reduce the amount of labeled A that binds to the antibody.

The term “treatment”, as used in the context of the invention refers to prophylactic, ameliorating, therapeutic or curative treatment.

The term “subject” is herein defined as vertebrate, particularly mammal, more particularly human. For purposes of research, the subject may particularly be at least one animal model, e.g., a mouse, rat and the like. In particular, for treatment of HBV infection and/or HBV-linked diseases, the subject may be a human infected by HBV.

A person skilled in the art will appreciate that the present invention may be practiced without undue experimentation according to the method given herein. The methods, techniques and chemicals are as described in the references given or from protocols in standard biotechnology and molecular biology text books.

### EXAMPLES

A person skilled in the art will appreciate that the present invention may be practiced without undue experimentation according to the methods given herein. The methods, techniques and chemicals are as described in the references given or from protocols in standard biotechnology and molecular biology textbooks. Standard molecular biology techniques known in the art and not specifically described were generally followed as described in Sambrook and Russel, Molecular Cloning: A Laboratory Manual, Cold Springs Harbor Laboratory, New York (2001).

### Example 1

### Materials and Methods

### Blood Samples

Human peripheral blood was obtained upon informed consent from an acute-recovered HBV patient (DSRB 2015/00354, Hepatitis B virus eradication and loss (HEAL) cohort study). Study protocols are approved by the National University of Singapore. All procedures performed in studies involving human participants complied with all relevant ethical regulations.

### Whole Antibody or Antibody Fragment Expression

Heavy and light chain variable regions of the selected antibody were cloned into plasmids with the respective human IgG (IgG1, IgG2, IgG3, IgG4) constant regions. Plasmids were transformed into E. Coli TOP10 by heat shock. A single colony was picked and grown overnight before plasmid extraction using EZNA®, an endo-free plasmid DNA mini kit (Omega Bio-Tek). Sequencing was performed to ensure no mutations were introduced. Transfection of heavy and light chain plasmids into HEK293 cells with polyethyleneimine (Sigma-Aldrich) was performed for the expression of full-length antibodies. Antibodies were purified from the culture supernatants using Protein G Sepharose™ 4 fast flow resin (GE Healthcare).

### HepG2-hNTCP Cells

HepG2-hNTCP cells were cultured in Dulbecco's Modified Eagle Medium (DMEM)+10% fetal bovine serum (FBS, Gibco)+2.5% puromycin in a T75 flask and maintained at 37° C. with 5% CO2 in a cell incubator.

### Virus Production

To produce HBV virus for infection, HepAD38 cells (Genotype D) were grown in a T75 flask and cultured in DMEM medium with tetracycline at 37° C. with 5% CO2 in a cell incubator. After two weeks, the medium was changed to DMEM medium without tetracycline (tet off). The cell supernatant was collected every three days. After collection, the virus was concentrated using 8% PEG 8000 before resuspension in PBS/10% FBS at 1/100 the volume. The virus mixture was aliquoted and stored at −80° C. for use.

### Capture ELISA

Briefly, MaxiSorp™ plates were coated with an anti-HBV capture antibody (Fitzgerald, PreS2) at 5 μg/ml overnight. Plates were blocked with 4% skim milk in PBS (SM-PBS) buffer at room temperature and then incubated with about 4×108 virions at 4° C. overnight. Plates were then incubated with antibodies (various concentrations) diluted in blocking buffer for 1 h. These antibodies can be supernatant from B cells during screening, or just antibody diluted to various concentrations for characterisation post antibody recovery. After that, each well was incubated with HRP-conjugated goat-anti-human IgG secondary antibody (Thermo Fisher Scientific, cat. #31413, diluted 3000×) for 1h, followed by incubation with TMB substrate for color development before H₂SO₄ addition to stop color development. OD₄₅₀ was recorded. Plates were washed four times with PBS between all incubation steps. Antibody coated wells with no virus added were included as negative controls.

### Sorting of Memory B Cells

Cryopreserved human PBMCs were thawed at 37° C. in complete media. Cells were washed with PBS, stained with Live/Dead™ blue viability dye (Thermo Fisher) and incubated in a cocktail of monoclonal antibodies (CD19 1:50-Biolegend, CD14: 1:50-Biolegend, CD3 1:100, CD27 1:50, CD38 1:50, IgG 1:50, IgM 1:50). B cells were washed then single cells were sorted into a 384-well plate using the BD FACSAria™ III cell sorter.

TABLE 1
List of antibodies used for cell sorting

| | | | | Catalog |
|---|---|---|---|---|
| Marker | Fluorochrome | Manufacturer | Clone | number |
| CD19 | BV510 | Biolegend | SJ25C1 | 363020 |
| CD3 | APC-Cy7 | Biolegend | OKT3 | 317342 |
| CD14 | APC-Cy7 | Biolegend | HCD14 | 325622 |
| CD27 | BV650 | Biolegend | 0323 | 302828 |
| CD38 | PE-Cy7 | Biolegend | HB-7 | 356608 |
| IgG | PerCP-Cy5.5 | BD Biosciences | G18-145 | 624060 |
| IgM | BUV496 | BD Biosciences | UCHB1 | 750366 |

### B-Cell Screening

Iscove's Modified Dulbecco's Medium (IMDM)+GlutaMAX™, supplemented with 10% ultra-low IgG and 1% penicillin/streptomycin, 50 μg/ml human transferrin and 5 μg/ml human insulin was used for culturing of human memory B cells. The medium is referred to as complete IMDM. The sorted human memory B cells were then resuspended in complete IMDM with the addition of an activation cytokine milieu, containing the following: 20 U/ml IL-2, 50 ng/ml IL-10, and 10 ng/ml IL-15 and 50 ng/ml monomeric soluble recombinant human CD40L. The memory B cells were cultured for 4 days under these conditions to allow for stimulation and expansion. After 4 days, the cells were pelleted and resuspended in new complete IMDM with the addition of a secretion cytokine milieu, containing the following: 20 U/ml IL-2, 50 ng/ml IL-10, 10 ng/ml IL-15, and 50 ng/ml IL-6. This was to promote differentiation to antibody-producing plasmablasts. Following another 3 days of culture, the cells were pelleted and lysed using the QuickExtract™ RNA extraction kit (Lucigen). The supernatants were harvested and used for ELISA screening. All cytokines were purchased from Cell Guidance Systems Ltd, UK.

### Antibody Sequence Recovery and Synthesis

The cell lysates, corresponding to the positive hits from the ELISA screening, were then used for downstream PCR and NGS analysis. cDNA was generated using the Maxima™ H Minus cDNA Synthesis Master Mix (Thermo FisherScientific) using 5 μL of extraction buffer per 10 μL reaction as per the manufacturer's protocol. Two microliters of cDNA products were directly used in 20 μL reactions with Platinum™ Taq Master Mix as per the manufacturer's protocol for separate amplification of the heavy and light chain variable regions. Illumina adaptor and barcode sequences were added by further PCR with Q5@ Hot Start Hi-Fidelity Master Mix and the PCR products purified with AMPure XP magnetic beads. Purified PCR products were pooled to obtain a 4 nM library and sequenced on the Illumina MiSeq with a 2× 300 bp kit with 25% PhiX spike-in. Individual reads were separated and corresponding germline sequences were retrieved from IMGT, the international ImMunoGeneTics database. The individual heavy and light sequences obtained were sent to Twist Biosciences for plasmid synthesis.

### Sodium Dodecyl Sulfate-Polyacrylamide Gel Electrophoresis (SDS-PAGE)

Gel electrophoresis was carried out with a 12%/15% polyacrylamide resolving gel to visualize protein sizes under reducing or nonreducing conditions. 5 μg of protein mixed with reducing or a nonreducing dye, incubated at 95° C. for 10 min was loaded onto separate lanes. Protein ladder (Thermo Fisher Scientific, cat. #26619) was loaded as a reference. Coomassie Brilliant Blue was used for gel staining before imaging on a gel imager (Bio-Rad).

### Western Blot

Briefly, protein bands separated on SDS-PAGE were transferred onto a polyvinylidene fluoride (PVDF) membrane. The membrane was blocked in 5% skim milk in PBST for 1 h, before incubation with 10 μg/mL primary antibody or control antibody. Next, the membrane was incubated with goat-anti-human IgG-HRP (Thermo Fisher Scientific, cat. #31413, diluted 10,000×). Three washes for 10 min each in PBST were conducted after step. WesternBright® ECL (Advansta) was added for protein visualization in the darkroom. Images were taken with an x-ray film (Advansta).

### HBV Neutralization Assay

Briefly, 5×10⁴ HepG2-hNTCP cells were seeded into a 96 well plate for neutralization, 1×10³ GE/cell of HBV and the respective antibody was mixed and inoculated with HepG2-hNTCP cells in the presence of 4% PEG 8000. After 20 h of incubation at 37° C., with 5% CO2, cells were thoroughly washed three times with 1×PBS and maintained in infection medium. Medium change was performed every two days. On day seven, supernatant from individual wells was harvested and used for the analysis of secreted HBsAg (Bio-Rad-Monolisa™ HBsAg kit) and HBeAg (BioRad-Monolisa™ HBeAg kit). The cells were harvested by trypsinization for analysis of intracellular HBcAg (Thermo Fisher Scientific, cat. #MA1-7606) via flow cytometry.

### Flow Cytometry (HBV HBcAg)

Harvested HepG2-hNTCP cells were fixed and permeabilized using the fixation/permeabilization solution kit (BD Biosciences cat. 554714) following kit instructions. Cells were incubated with HBcAg (Thermo Fisher Scientific, cat. #MA1-7606) at 1000× dilution at 4° C. for 1 h in permeabilization buffer. After washing, cells were stained with AlexaFluor®-647 conjugated goat-anti-mouse IgG antibodies (Invitrogen cat. #A21235) at a 300× dilution. Uninfected cells were stained with the same protocol as a control. All sample data were acquired using the Attune N×T flow cytometer and data were analyzed using FlowJo software.

### Affinity Determination of Antibodies

Antibody kinetics and affinity were measured using an Attana Cell A200 (Attana AB) at 25° C. An LNB carboxyl sensor chip was used for the experiments. Activation of the chip was carried out with sulfo NHS/EDC (Amine coupling kit, Attana) and only the experimental chip was saturated with rHBsAg. Ethanolamine was added to deactivate the chip surface. PBS was used as a running buffer for subsequent injections. Antibody dilutions were optimized together with regeneration conditions and randomly tested by the robotic arm. Antibody injections were applied as 105 s pulses at a flow rate of 20 μL min-1 and dissociation was monitored for 300 s. Regeneration of the chip was done by two 10 s pulses of 20 mM Glycine, pH 5.0. Curve fitting and data analysis were performed using the trace drawer software.

### HBV DNA Quantification

HBV DNA was isolated following the Qiagen QIAampR DNA Blood Kit instructions.

Briefly, lysis buffer together with Proteinase K was added to the sample at 57° C. for 15 minutes. The sample was spun through the provided column and washed thoroughly, before elution. 5 μL of the eluted sample was used for qPCR. qPCR was performed with an SYBR® Green qPCR (Thermo Fisher Scientific, cat. 4309155) using HBV DNA specific primers: 1) Virus-For and 2) Virus-Rev using ABI7500 Fast Real-Time system instrument (Applied Biosystems). Viral DNA copy number was calculated based on a standard curve generated from a sample with known copy numbers.

Immunoprecipitation Assay 100 μl of HBV was incubated with an isotype-controlled antibody and Protein G Sepharose for 2 h at 4° C. for pre-clear. The sample was divided and immunoprecipitated with the antibody of interest, positive control (Fitzgerald PreS2 antibody), or PBS (negative control) overnight at 4° C. Next, protein G beads were added and rotated for 2 h at 4° C. The beads were spun down washed 3 times with 0.1% PBST. HBV DNA extraction was performed for quantification.

### Multiplex Immunoassay

Using the Bio-Plex™ 200 platform (BioRad), a multiplex HBsAg epitope mapping was developed targeting anti-HBs epitopes across five HBsAg domains [Walsh, R. et al., Liver Int 39:2066-2076 (2019); Hyakumura, M. et al., J Virol 89:11312-11322 (2015)]. Individual fluorescently identified magnetic beads were pre-conjugated with anti-HBs mAbs and plexed together. Polyclonal phycoerythrin-conjugated antibodies were used for detection. The epitope specificity of the selected multiplex anti-HBs mAbs to particular HBsAg domains spans residues 99-160 in the ‘a’ determinant and target the loop 1 and loop 2 regions. Using this method, the effect of anti-HBs antibodies on the HBsAg epitope profile was investigated by pre-incubating mAb11 with an HBsAg wildtype reference, prior to testing in the multiplex immunoassay. On analysis, alterations in the epitope profile mapping between HBsAg only and HBsAg pre-incubated with mAb11 are compared. Reduced epitope recognition at both loops 1 and 2 epitopes is considered significant, ‘inducing’ a clearance profile.

The conjugated mAbs have the following specificities, mAbs 5, 6, and 10: loop 1; mAbs 7, 8, 11, 12, 16, and 17: loop2. The origin of the mAbs is acknowledged by Hyakumura et al., [Hyakumura, M. et al., J Virol 89:11312-11322 (2015)], and an illustration of the antibody binding sites has been published by Walsh et al., [Walsh, R. et al., Liver Int 39:2066-2076 (2019)]. The 95% confidence interval (CI) for the normal range of variation of epitope recognition from the reference backbone was established as ±0.5-fold change. A fold change <0.5 was considered insignificant reflecting the normal variance of this assay. Positive fold changes (>0.5-fold) and negative fold changes (>0.5-fold) corresponded to a gain or reduction of epitope binding, respectively. A threefold reduction was considered a complete epitope knockout.

Linear epitope binding A direct ELISA protocol was carried out coating HBsAg peptides (50 ng/well): (loop1, T-C-T-T/I-P-A-Q-G-N/T-S-M-F-P-S-C, loop 2, C-T-K-P-T/S-D-G-N-C-T) in phosphate-buffered saline (PBS). These were bound to microtiter plates (Maxisorp, Nunc) at 4° C. overnight, and then each well was blocked in 5% skim milk in PBS Tween 20 (0.05%) at room temperature for 2 hours. Antibodies (mAb11) were incubated at an appropriate dilution in PBS for 1 hour at room temperature. Bound antibody was detected with an anti-human immunoglobulin (Ig) antibody conjugated to horseradish peroxidase. After several washing steps, antibody binding was detected by the addition of ABTS [2, 2′-azinobis (3-ethylbenzthiazoline-6-sulfonic acid); Sigma] and H₂O₂ in a citrate phosphate buffer. OD's were read at 410 nm after 30 minutes.

### Generation of Human Liver Chimeric Mice

Fah^{-/-}/Rag2^{-/-}/IL2rg^{-/-}(FRG) triple knockout mice purchased from Yecuris Corporation were maintained with (2-(2-nitro-4-trifluoromethylbenzoyl)-1,3-cyclohexanedione) (NTBC) at a concentration of 16 mg/l. All mice were bred and kept under specific pathogen-free with 12-hour light/12 dark cycle conditions in the Biological Resource Centre, Agency for Science, Technology and Research, Singapore in accordance with the guidelines of the Agri-Food and Veterinary Authority and the National Advisory Committee for Laboratory Animal Research of Singapore. Briefly, 4-6-week-old mice were intrasplenically injected with 1×10⁶ primary human hepatocytes (PHH) (Lonza). Following surgery, NTBC was withdrawn through a series of cycling conditions to facilitate mouse hepatocyte cell death. Repopulation of human hepatocytes generally takes around 2-3 months whereby the production of ˜5 mg/ml human albumin (hALB) in mouse serum represents ˜70% humanization of the liver indicative of mice being experimentally ready.

Quantification of hALB Production

Production of hALB in mice was measured by ELISA according to the manufacturer's instructions (Bethyl Laboratories Inc). Briefly, serum isolated from mouse blood was diluted 10⁶ times with sample diluent followed by the kit's protocol.

### Quantification of HBsAg in Mice Serum

Mice serum was diluted 10× in PBS before quantification of HBsAg according to manufacturer's instructions (HBsAg CLIA, #cat CL0310-2, Autobio). Briefly, serum from the different time points was incubated with an enzyme conjugate in a pre-coated ELISA plate for an hour. Six washes were performed with PBS followed by the addition of a substrate cocktail provided by the kit. HBsAg was detected by luminescence using a Tecan plate reader. The kit also provides a series of HBsAg standards in International Units per ml (IU/ml) to generate a standard curve for HBsAg quantification.

### Histological Analysis of Mice Organs

All mouse livers, kidneys, and spleens were collected and processed for histology at the endpoint. Briefly, all tissues were fixed in 10% formalin before paraffin-embedment. All tissue sections were rehydrated and stained with H&E (ThermoFisher Scientific) according to the manufacturer's protocol. Liver sections were also stained with hFAH (ab140167, Abcam) and purified recombinant HBcAg (MA1-7607, ThermoFisher Scientific) antibodies by immunohistochemistry according to the manufacturer's protocol. All images were captured by the ZEISS Axio Scan.Z1 and processed using the Zen lite software.

### Determining Plasma Antibody Concentration

A MaxiSorp™ ELISA plate was coated with 1 μg/ml protein A overnight. The plate was washed, and the wells were blocked with 4% milk-PBS. Serum samples were diluted 1/50 in 4% milk/PBS and added to the wells for 1 h. The plate was washed thoroughly before the addition of mouse anti-human IgG-HRP secondary antibody for 1h. After washing the plate 3 times, TMB was added to allow HRP signal development for 5 minutes before the signal was quenched by H₂SO₄. The plate was read at a wavelength of 450 nm to determine absorbance. A standard curve was set up on the same plate to allow antibody concentration determination.

### Example 2

### HBV Specific Human Antibody Isolation

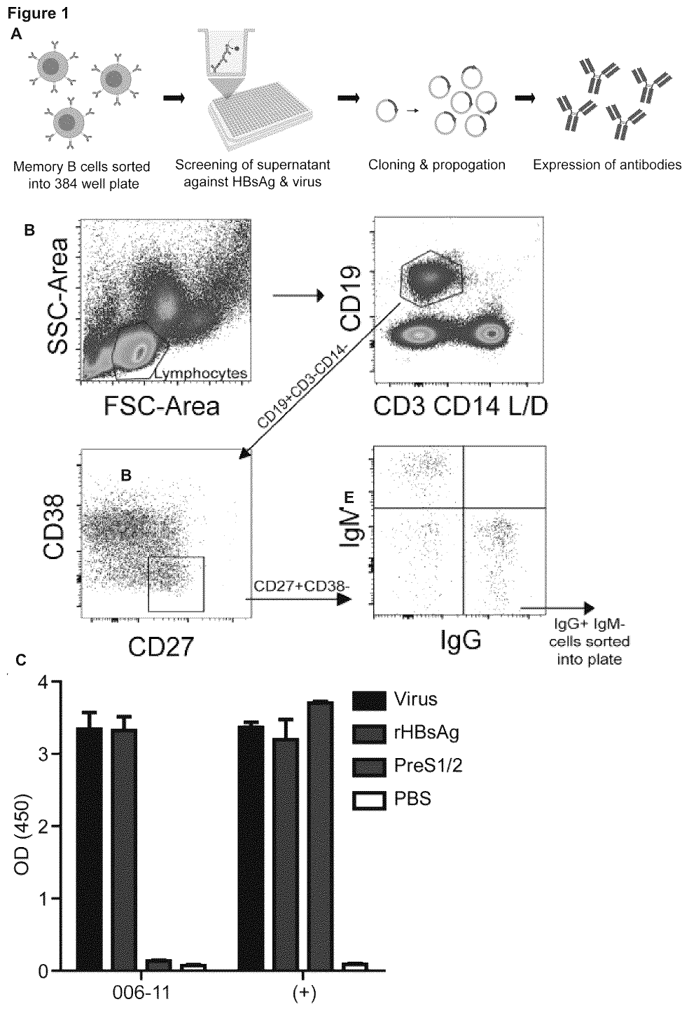

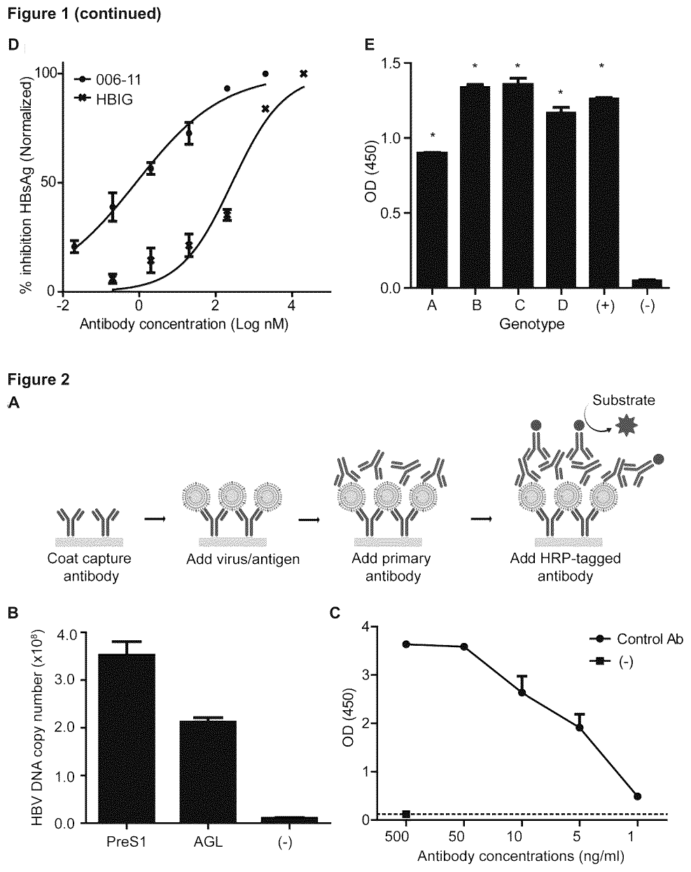

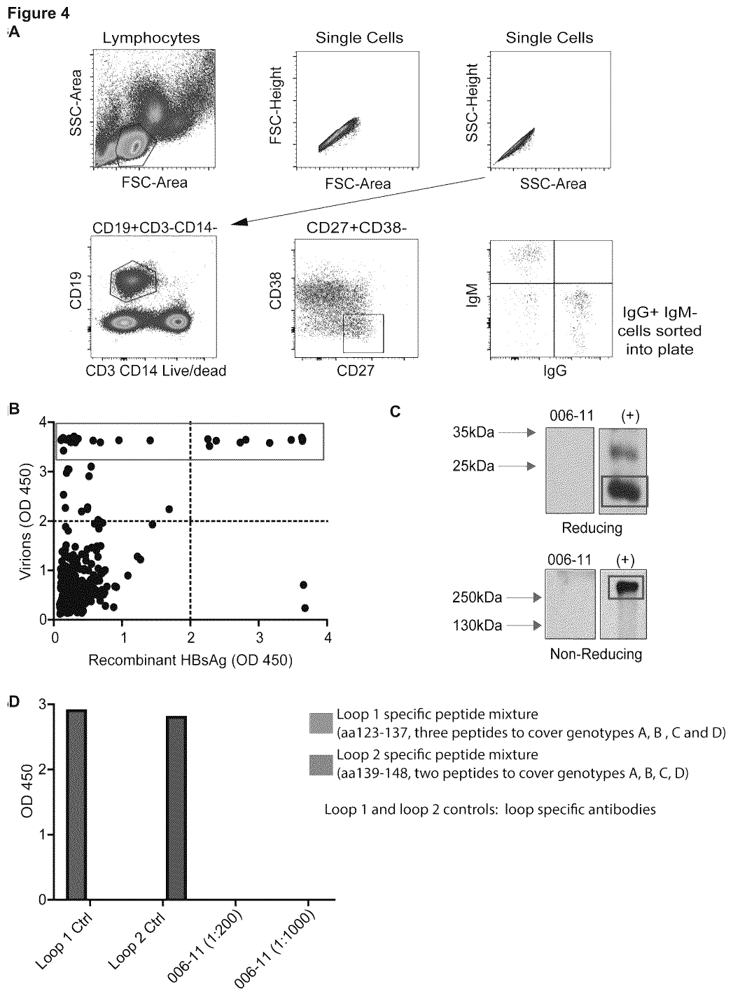

To facilitate the discovery of highly potent anti-HBsAg antibodies, a capture ELISA allowing for an unbiased screen against the whole virus (FIG. 1A, FIG. 2A-C) was developed. This assay was used in conjunction with a single-cell antibody discovery method for the isolation of anti-HBsAg specific antibodies from PBMC's obtained from an HBV resolved individual. A schematic of the discovery method can be seen in FIG. 1A. We isolated a fully human IgG1 monoclonal antibody termed mAb006-11 which showed binding only to recombinant small-HBsAg (referred to as HBsAg) and HBV virions (FIG. 1B, FIG. 4A-B).

Next, the neutralizing potential of mAb006-11 was tested in-vitro by employing HepG2/NTCP cells which are susceptible to HBV infection. mAb006-11 was found to be a potent neutralizer due to its ability to prevent HBV infection at nanomolar antibody concentrations as determined by quantifying secreted HBsAg in the supernatant (FIG. 1C). mAb006-11 was observed to be a superior neutralizing antibody compared to HBIG with an EC₅₀ value in the low nanomolar range. As mAb006-11 was determined to be a potent neutralizer with the ability to prevent an active infection from forming in an in-vitro setting, further examination of the cross-binding breadth of mAb006-11 showed that it has a similar binding profile to all the four major genotypes A, B, C and D out of the ten known HBV genotypes (FIG. 1D, Table 2).

TABLE 2
Amino acid sequence of HBsAg (Genotypes)

| | | SEQ ID |
|---|---|---|
| Genotype | Amino acid sequence | NO: |
| A (adw2) | MENITSGFLGPLLVLQAGFFLLTRILTIPQ | 11 |
| | SLDSWWTSLNFLGGSPVCLGQNSQSPTSNH | |
| | SPTSCPPICPGYRWMCLRRFIIFLFILLLC | |
| | LIFLLVLLDYQGMLPVCPLIPGSTTTSTGP | |
| | CKTCTTPAQGNSMFPSCCCTKPTDGNCTCI | |
| | PIPSSWAFAKYLWEWASVRFSWLSLLVPFV | |
| | QWFVGLSPTVWLSAIWMMWYWGPSLYSIVS | |
| | PFIPLLPIFFCLWVYI | |
| B(adw) | MENIASGLLGPLLVLQAGFFLLTKILTIPQ | 12 |
| | SLDSWWTSLNFLGGTPVCLGQNSQSQISSH | |
| | SPTCCPPICPGYRWMCLRRFIIFLCILLLC | |
| | LIFLLVLLDYQGMLPVCPLIPGSSTTSTGP | |
| | CKTCTTPAQGTSMFPSCCCTKPTDGNCTCI | |
| | PIPSSWAFAKYLWEWTSVRFSWLSLLVPFV | |
| | QWFVGLSPTVWLSVIWMMWFWGPSLYNILS | |
| | PFMPLLPIFFCLWVYI | |
| C (adr) | MENTASGFLGPLLVLQAGFFLLTRILTIPQ | 13 |
| | SLDSWWTSLNFLGGAPTCPGQNSQSPTSNH | |
| | SPTSCPPICPGYRWMCLRRFIIFLFILLLC | |
| | LIFLLVLLDYHGMLPVCPLLPGTSTTSTGP | |
| | CKTCTIPAQGTSMFPSCCCTKPSDGNCTCI | |
| | PIPSSWAFARFLWEWASVRFSWLSLLVPFV | |
| | QWFVGLSPTVWLSVIWMMWYWGPSLYNILS | |
| | PFLPLLPIFFCLWVYI | |
| D (ayw) | MENITSGFLGPLLVLQAGFFLLTRILTIPQ | 14 |
| | SLDSWWTSLNFLGGTTVCLGQNSQSPTSNH | |
| | SPTSCPPTCPGYRWMCLRRFIIFLFILLLC | |
| | LIFLLVLLDYQGMLPVCPLIPGSSTTSTGC | |
| | RTCMTTAQGTSMYPSCCCTKPSDGNCTCIP | |
| | IPSSWAFGKFLWEWASARFSWLSLLVPFVQ | |
| | WFVGLSPTVWLSVIWMMWYWGPSLYSILSP | |
| | FLPLLPIFFCLWVYI | |

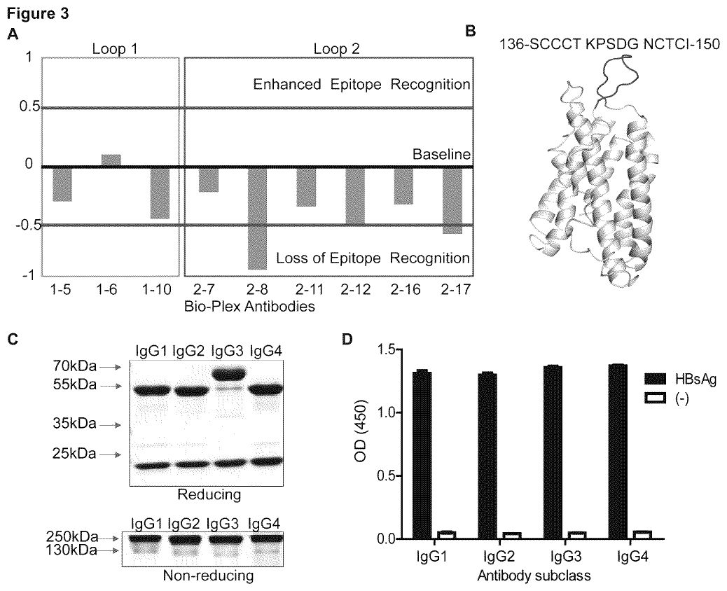

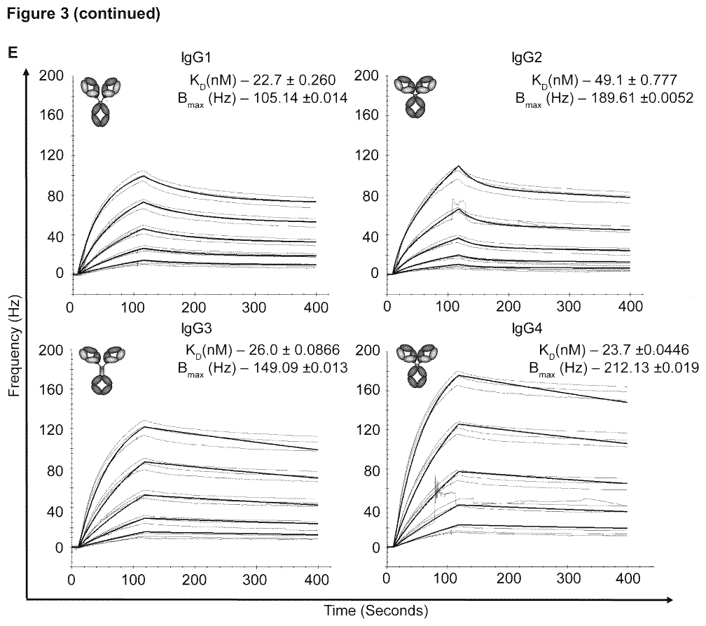

It was observed that mAb006-11 lost binding activity when HBsAg was chemically reduced (FIG. 4C). Further analysis using the Bio-Plex® assay showed that mAb006-11 blocked access to epitopes only in the loop 2 region (FIG. 3A). Additionally, when mAb006-11 was testing for its binding activity against loop 1 and 2 linear peptides, it showed no binding ability (FIG. 4D). To visualize the predicted binding site for mAb006-11, a 3D structure of HBsAg Genotype A was predicted using the I-TASSER server, and the binding region is highlighted in red (FIG. 3B). This data suggests that mAb11 binds to a highly denaturation sensitive, conformational epitope found on HBsAg.

### Example 3

A Functional Comparison of mAb006-11-IgG Subclasses

As mAb006-11-IgG1 was found to be a potent neutralizer, the variable region of the mAb006-11 heavy chain was engineered onto the backbone of the four principal IgG subclasses. This was done to test if subclass differences affected the binding and neutralizing ability of mAb006-11. FIG. 3C shows the reducing and non-reducing gels, of the four expressed subclass antibodies. The presence of a single band on the non-reducing gel and two bands on the reducing gel of approximately 150 kDa and 50 kDa, 25 kDa respectively indicates that the antibodies were expressed properly. The larger heavy chain band seen for IgG3 is due to this subclass having a longer hinge region compared to the other subclass. The binding abilities of the IgG2, IgG3, and IgG4 subclasses were verified against recombinant HBsAg via ELISA and showed similar binding to its IgG1 subclass (FIG. 3D).

A detailed biophysical analysis of the interaction between the four principal IgG subclasses of mAb006-11 and HBsAg utilizing the quartz crystal microbalance (QCM) technology was carried out. The calculated dissociation equilibrium constant (K_{D}) for the four molecules was 22.7 nM, 49.8 nM, 26.0 nM and 23.7 nM for IgG1, IgG2, IgG3, and IgG4 respectively (FIG. 3E). In terms of affinity, minimal differences in affinity were observed between IgG1, IgG3, and IgG4 while IgG2 showed a slightly lower affinity. The other parameter which was analyzed for the four principal subclasses was the Bₘₐₓ value which is 105.14 Hz, 189.61 Hz, 149.09 Hz, and 212.12 Hz for IgG1, IgG2, IgG3, and IgG4 respectively (FIG. 3E). The Bₘₐₓ value indicates the maximum number of antibody molecules bound to the antigen. The slightly smaller size for the IgG2 and IgG4 subclasses and the highly flexible hinge region for IgG3 might thus explain the higher Bₘₐₓ value for these molecules compared to the IgG1 subclass.

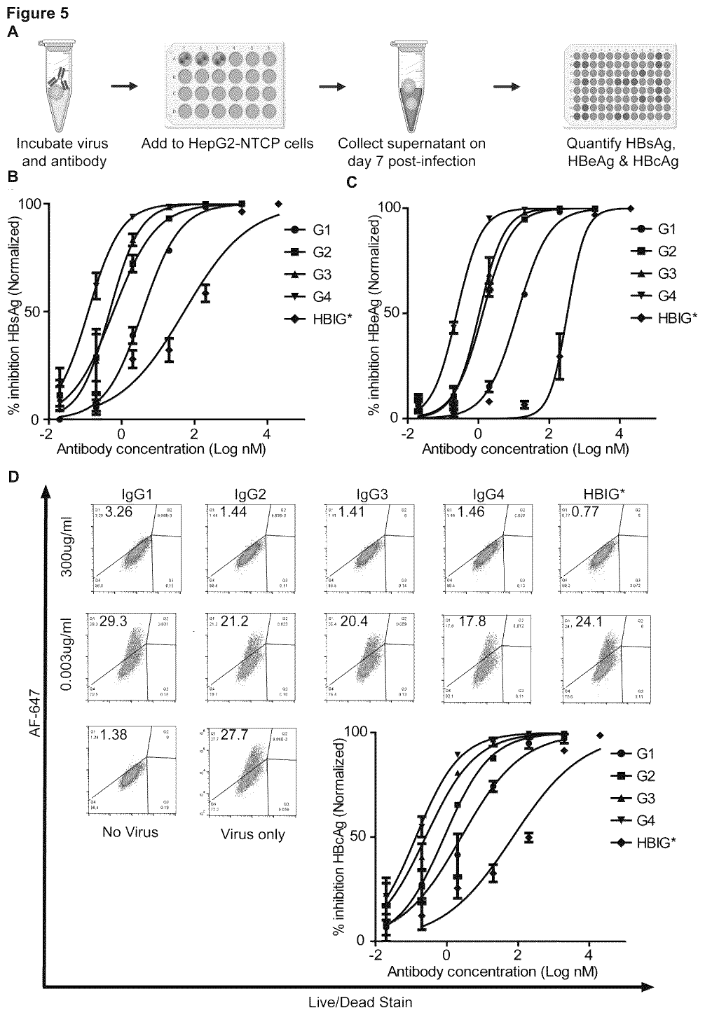

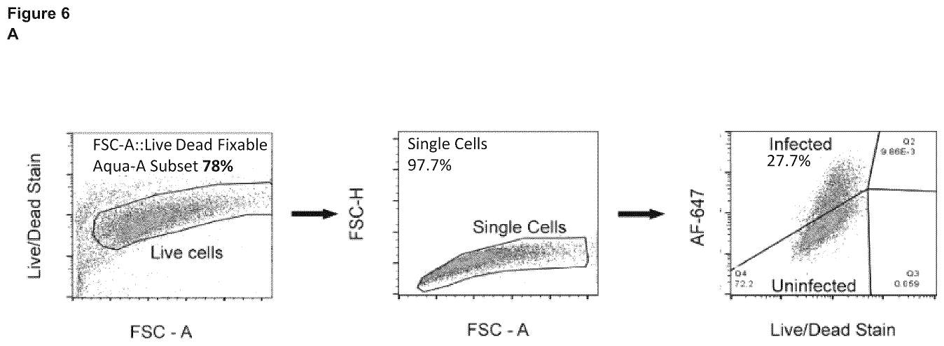

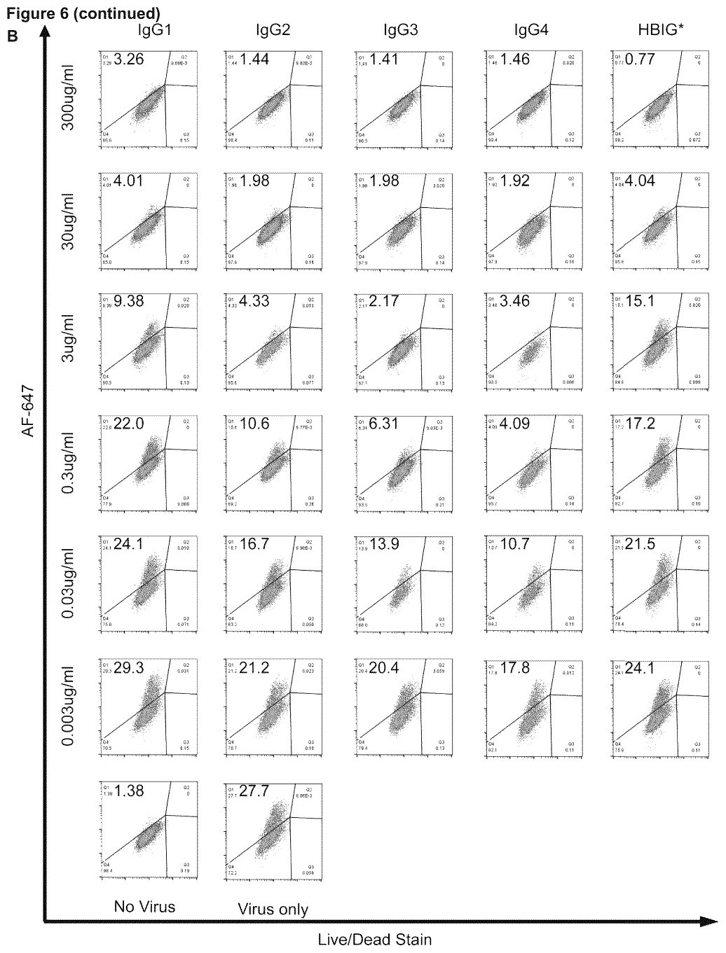

The neutralizing potential of four subclasses was next compared via an in-vitro neutralization assay, a schematic of the assay can be seen in FIG. 5A. FIGS. 5B, C, and D compare the neutralizing potential of the four antibody subclasses by quantifying secreted HBsAg, HBeAg, and intracellular HBcAg respectively. All three data sets indicate that the mAb006-11-IgG4 subclass is the most potent neutralizer with an EC₅₀ of 0.242 nM (HBeAg data). This was followed by mAb006-11-IgG3 (EC₅₀-1.05 nM), IgG2 (EC₅₀-1.325 nM) and finally IgG1 (EC₅₀-12.63 nM). All the subclasses of mAb006-11 were also observed to have a more potent neutralizing potential compared to HBIG (EC₅₀-329.9 nM).

Analyzing the neutralization potential and biochemical characteristics of the difference subclasses, mAb006-11-IgG4 is the best neutralizer due to its high binding affinity and high Bₘₐₓ. Comparing IgG2 and IgG3 which are the next best neutralizer with only a slight difference in neutralizing potential, the inventors observed that IgG2 has a higher Bₘₐₓ but lower affinity while IgG3 has a slightly lower Bₘₐₓ but a higher affinity. This means that both these characteristics are essential in determining the neutralizing potential of the antibody, the K_{D} value determines how fast the antibody binds and how long it stays on, while the Bₘₐₓ value determines the total number of antibody molecules decorated on the antigen target. Both these factors play an important role in deciphering the antagonizing potential of an antibody.

### Example 4

mAb006-11 as a Prophylactic and Therapeutic Strategy Against HBV In Vivo

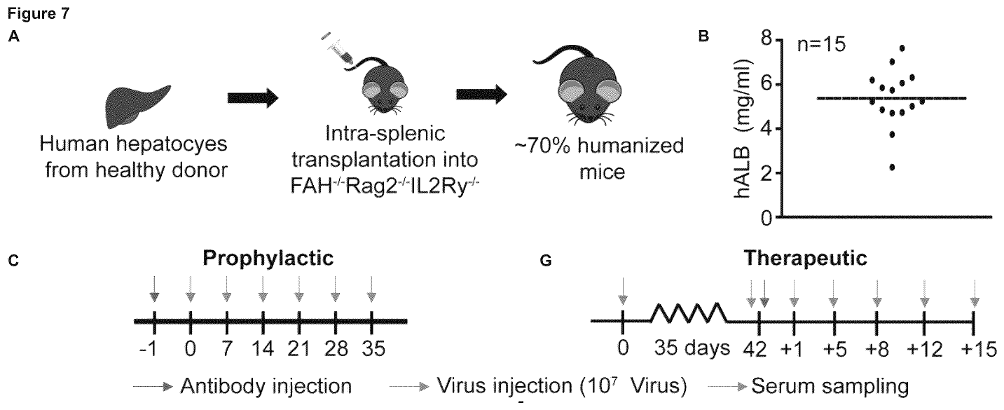

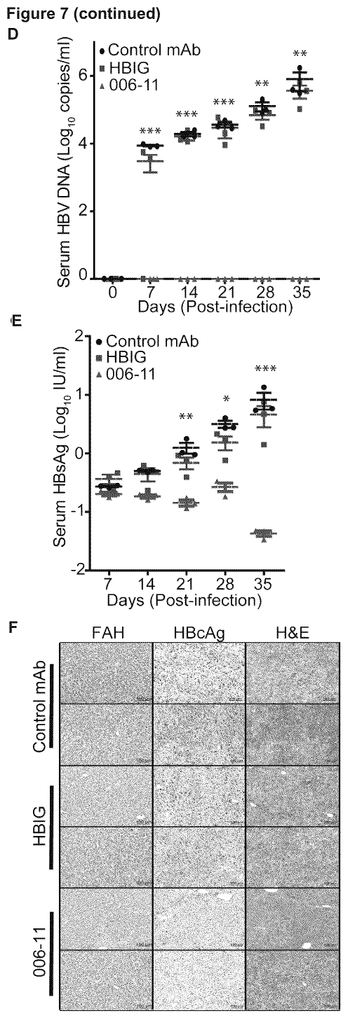

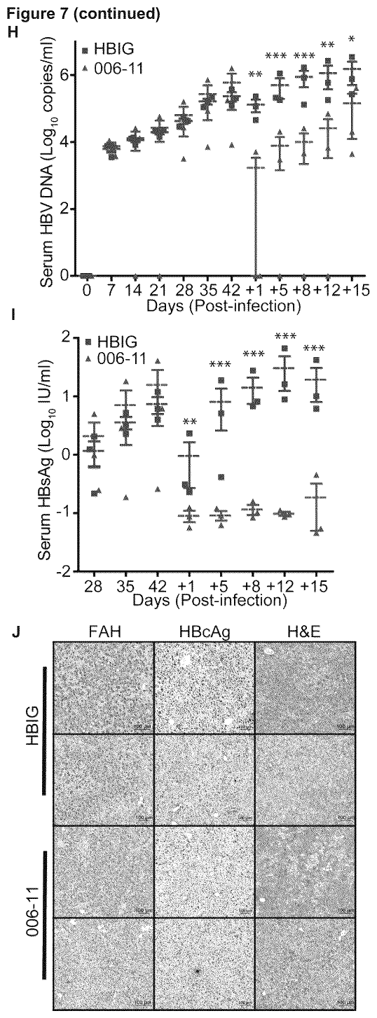

The prophylactic and therapeutic potential of mAb006-11 was investigated in human liver chimeric (HuFRG, ˜70% of human hepatocytes) mice [Azuma, H. et al. Nat Biotechnol 25, 903-910 (2007)], which support HBV infection. A schematic illustration of the humanization of HuFRG mice is shown in FIG. 7A. Mice are experimentally ready for HBV inoculation when hALB levels achieved ˜5 mg/ml as shown in FIG. 7B.

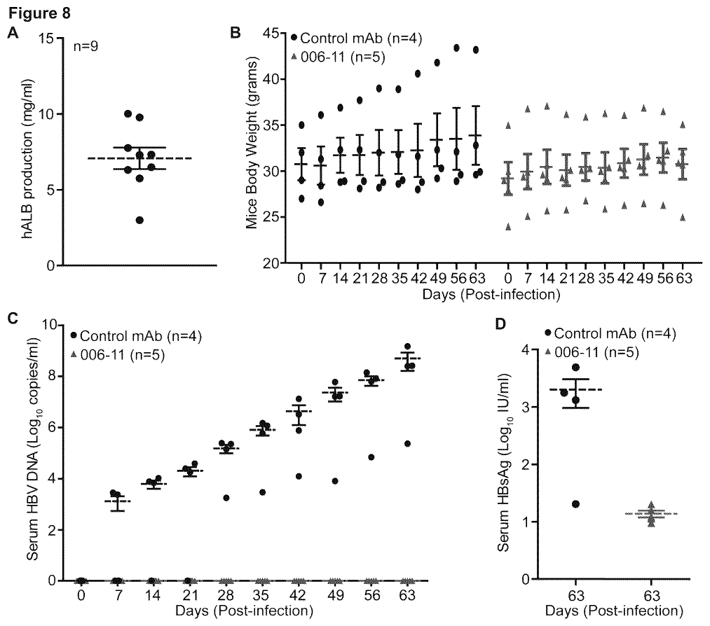

In the initial experimental setup, we evaluated the prophylactic efficacy of mAb006-11 in HBV-infected HuFRG mice. A single dose (100 μg per mouse) of control mAb or mAb006-11 was administered intraperitoneally to each mouse one day before HBV infection followed by measurement of HBV DNA weekly up to 63 days post-infection (dpi). Both mice groups did not exhibit any physical abnormalities or changes in body weight (FIG. 8B), indicating that they were healthy. However, drastic differences in HBV DNA and serum HBsAg levels were observed between the two groups. The treated group (mAb006-11) displayed HBV DNA levels below the detection limit whereas the control mAb group exhibited increasing DNA levels of up to ˜9 Log copies/ml (FIG. 8C). Also, mice treated with mAb006-11 portrayed HBsAg levels ˜3 Logs IU/ml lower at the endpoint when compared to the control group indicating complete viral entry blockade (FIG. 8D).

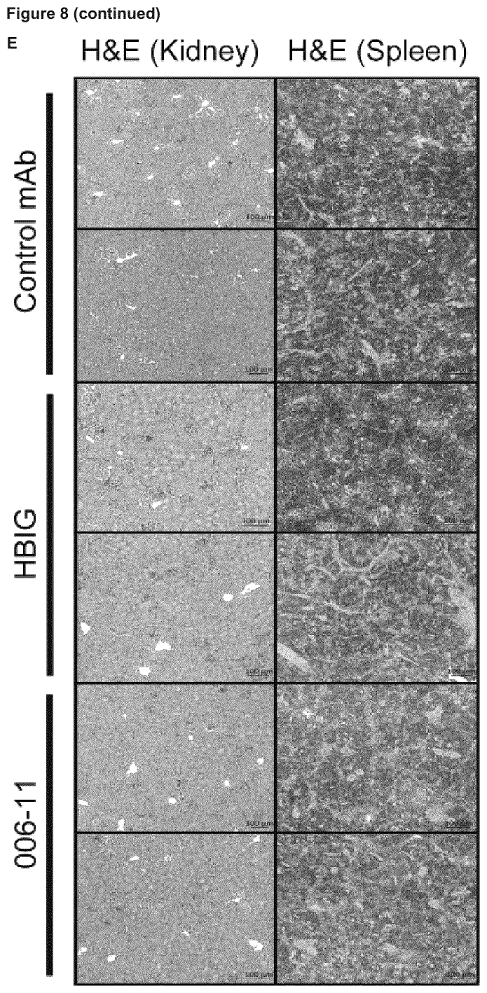

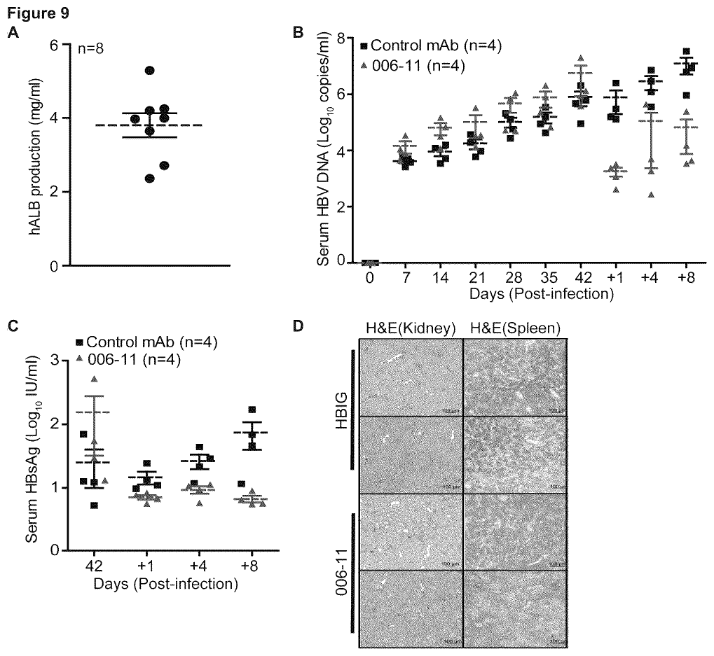

The effects of mAb006-11 at a therapeutic level were investigated. HBV infection was first established in HuFRG mice for 42 dpi before the administration of a single dose (300 μg per mouse) of control mAb or mAb006-11. Both HBV DNA and HBsAg levels were measured at 1, 4, and 8 days post-antibody injection. At endpoint, a ˜3 Logs and ˜2 Logs reduction was observed in HBV DNA and HBsAg levels respectively in mice injected with mAb006-11 compared to the control group (FIG. 9B, 9C).

Having observed potent prophylactic and therapeutic efficacy of mAb006-11 in-vivo, the inventors next compared the prophylactic efficacy of a single dose of mAb006-11 against HBIG and control mAb. A single antibody dose (100 μg per mouse) was similarly administered intraperitoneally to HuFRG mice one day before being challenged with HBV (FIG. 7C). Nine HuFRG mice were randomly injected with either a control mAb, HBIG, or mAb006-11 at the same concentration and were monitored for over 35 days. Both the HBIG and control groups showed a steady increase in HBV DNA for 35 dpi (˜6 Log copies/ml) whereas DNA levels remained undetectable in the mAb006-11 group (FIG. 7D). In addition, serum HBsAg levels for both control mAb and HBIG groups were measured at around 1 Log IU/ml which is ˜100 times higher compared to mice treated with mAb006-11 at 35 dpi (FIG. 7E). At the endpoint, mice liver, spleen, and kidneys were harvested for histological analyses. All mice liver sections stained positive for human fumarylacetoacetate hydrolase (hFAH) by immunohistochemistry (IHC) indicating robust repopulation of human hepatocytes (FIG. 7F). Furthermore, intracellular HBcAg was detected (evidence of active infection) by IHC in liver sections of both control mAb and HBIG groups but not in mAb006-11 treated mice. Lastly, H&E stains of all mouse liver (FIG. 7F) sections including spleen and kidneys (FIG. 8E) exhibited normal organ morphologies with no adverse toxicity. This data demonstrated the prophylactic efficacy of mAb006-11 achieving complete virus blockade, thereby preventing the establishment of an active infection.

Next, the therapeutic potential of mAb006-11 against an established infection was evaluated (FIG. 7G). Six mice measuring an average of ˜5 Log copies/ml HBV DNA at 42 dpi were randomly administered with a single dose (300 μg per mouse) of the respective antibodies. Although mice treated with HBIG showed a subtle decrease in HBV DNA and HBsAg levels after 24 hr, both levels rebounded to initial levels at 5 days post-injection and continued to rise following time (FIG. 7H, 7I). On the other hand, mAb006-11-treated mice exhibited a more robust decrease in both HBV DNA (˜2 Log copies/ml) and HBsAg levels (˜2 Logs IU/ml) after 1 day post-injection suggesting that mAb006-11 is simply more efficient in blocking HBV replication compared to HBIG (FIG. 7H, 7I). More importantly, virus titer rebound was kept at a minimum whereas HBsAg production was maintained at close to undetectable levels (FIG. 7H, 7I). Similarly, histological analysis of mice liver sections in both groups displayed strong hFAH staining. In contrast, HBcAg staining was more abundant in liver sections of HBIG treated mice compared to mAb006-11 treated ones (FIG. 7J). H&E staining of mice liver, kidney, and spleen sections did not exhibit any abnormalities (FIG. 8J, FIG. 9D).

### Example 5

Comparison of mAb006-11 to Known Anti-HBV Antibody HBC34

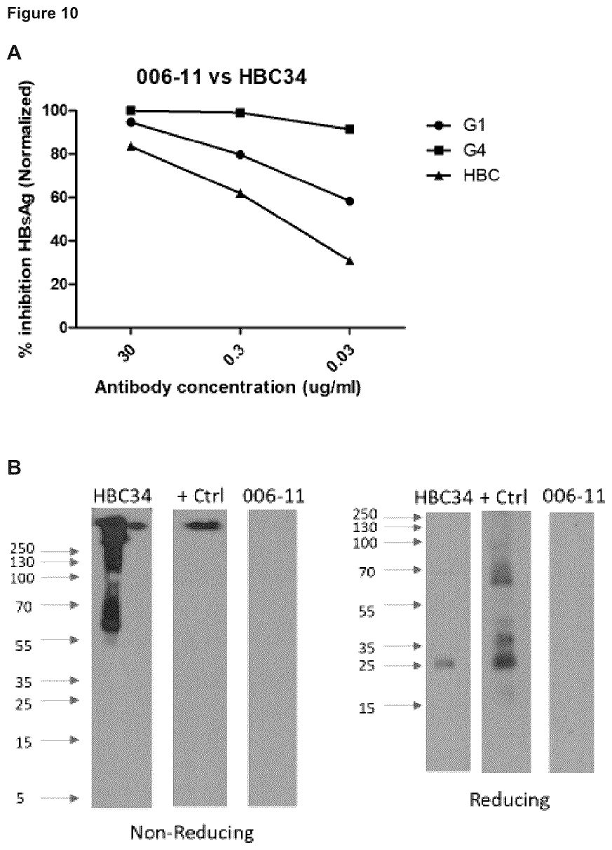

The neutralization abilities of mAb006-11 and HBC34 (WO2017/060504 A1) were compared using the in-vitro assay described in Example 1. The data shows both the G1 and G4 versions of mAb006-11 were superior in neutralizing ability when compared to HBC34 (FIG. 10A). At a concentration of 0.03 μg/ml, mAb006-11G1 neutralization was about 92% whereas for HBC34 the neutralization was about 29%. When comparing the binding epitope of mAb006-11 and HBC34, it was noted that mAb006-11 does not bind to HBV S-antigen under reducing or non-reducing conditions, however this was not the case with HBC34 which is shown to have strong binding to S-antigen under non-reducing conditions and weak binding under reducing conditions (FIG. 10B). This indicates that mAb006-11 is binding to an epitope which is conformation dependent.

### SUMMARY

Disclosed is the isolation, detailed characterization, and in-vivo testing of a fully human monoclonal antibody with potent neutralizing efficacy. The mAb006-11 heavy and light chains belong to the IgG1 subclass and Lambda light-chain group. Previous studies that have reported anti-HBs antibodies utilized either recombinant or synthetic proteins for their discovery efforts. As such, this is the first study in which direct screening against the whole virus for isolation of naturally paired anti-HBs antibodies is reported. The direct screening methodology adopted has a clear advantage as it allows for non-biased isolation of antibodies against all three surface antigens in their native state found on the surface of the virus. This is essential as structural studies to understand possible differences between the presentation of HBsAg on recombinant sub-viral particles and virions have not been reported. In FIG. 1C, we present the neutralization potential of mAb006-11 and show its superiority compared to HBIG at preventing HBV infection in-vitro. mAb006-11 displayed a cross-binding ability to the four most common HBV genotypes (A, B, C, D) indicating that it is binding to a conserved epitope and can potentially provide broad protection.

Subclass engineering was carried out to determine if the structural differences between the subclass affected the mAb006-11's binding and neutralizing potency. While subclass differences seemed to have little impact on the binding affinity of mAb006-11 subclasses to HBsAg, as noted by their nanomolar dissociation constant (FIG. 3C) for the three most commonly seen subclasses, the Bₘₐₓ values for the subclasses differed significantly which hint that structural differences are affecting the stoichiometry of antibody binding to HBsAg. The two smallest IgG subclasses (IgG2, IgG4) showed the highest Bₘₐₓ values followed by IgG3 which has a highly flexible hinge and finally IgG1. The neutralizing potential of the four subclasses was noted to be in the following order IgG4, IgG3, IgG2, and IgG1 (FIG. 5B, C, D). Neutralizing potency is closely correlated to the Bₘₐₓ value while affinity affects it to some degree, which indicates that both parameters are playing an essential role. Previous studies analyzing anti-HBs subclasses have reported that IgG1, IgG3 levels are the most dominant followed by IgG4 levels in convalescent and chronic patients. Another paper reported that anti-HBs IgG4 were found in immune complexes with poor clearing ability due to a lower affinity for Fc receptor engagement. This suggests that the IgG4 antibody response seen in chronic patients might be a protective mechanism to prevent an inflammatory response. The more potent neutralizing effect observed for mAb006-11-IgG4 might, therefore, be attributed to IgG4 molecules decorating the surface of virions more efficiently, thus preventing virus-receptor attachment and infection. This hints that IgG4 antibodies could function efficiently as an antagonist, preventing viral infection and spread. While IgG1 and IgG3 antibodies play an important role in activating downstream effector functions to clear an acute or chronic infection, an IgG4 subclass might be a better choice as a prophylactic.

Data presented herein shows that a single dose of mAb006-11 effectively prevented HuFRG mice from establishing an active HBV infection. This indicates that mAb006-11 not only potently inhibits viral entry at the frontline but also cleared virions efficiently from circulation. Similarly, when compared to HBIG, a commercially available prophylactic antibody used in clinical settings, mAb006-11 showed superior protection. Likewise, when used as a therapeutic intervention, mAb006-11 showed a potent reduction in both HBV DNA and serum HBsAg levels of 2-3 logs copies/ml and IU/ml respectively. More importantly, the antibody dosage of mAb006-11 used was significantly lower (3 mg/kg for prophylactic and 9 mg/kg for therapeutic) compared to other anti-HBs antibodies (15-20 mg/kg for prophylactic and 20 mg/kg for therapeutic with multiple doses) in vivo [Li, D. et al., Elife 6 (2017); Zhang, T. Y. et al., Gut 65:658-671 (2016)] which further substantiate our claim that mAb006-11 specifically targets an essential epitope required for viral entry, thus effectively inhibiting infection.

The monoclonal nature of mAb006-11 is another advantage compared to HBIG which is derived from purified human plasma and is plagued by issues such as limited availability, low specificity, and batch to batch variation. In contrast, a monoclonal anti-HBsAg would provide a stable and reproducible source for use in clinics.

### REFERENCES

Any listing or discussion of an apparently prior-published document in this specification should not necessarily be taken as an acknowledgement that such document is part of the state of the art or is common general knowledge.

Alavian, S. M., Carman, W. F. & Jazayeri, S. M. HBsAg variants: diagnostic-escape and diagnostic dilemma. J Clin Virol 57, 201-208 (2013).

Azuma, H. et al. Robust expansion of human hepatocytes in Fah−/−/Rag2-/−/112rg−/−mice. Nat Biotechnol 25, 903-910 (2007).

Both, L. et al. Monoclonal antibodies for prophylactic and therapeutic use against viral infections. Vaccine 31, 1553-1559 (2013).

Cerino, A., Bremer, C. M., Glebe, D. & Mondelli, M. U. A Human Monoclonal Antibody against Hepatitis B Surface Antigen with Potent Neutralizing Activity. PLoS One 10, e0125704 (2015).

Chen, K. L. et al. Hepatitis B virus reactivation and hepatitis in diffuse large B-cell lymphoma patients with resolved hepatitis B receiving rituximab-containing chemotherapy: risk factors and survival. Chin J Cancer 34, 225-234 (2015).

Crespo, G., Marino, Z., Navasa, M. & Forns, X. Viral hepatitis in liver transplantation. Gastroenterology 142, 1373-1383 e1371 (2012).

Eren, R. et al. Human monoclonal antibodies specific to hepatitis B virus generated in a human/mouse radiation chimera: the Trimera system. Immunology 93, 154-161 (1998).

Ferrone, S., & Dierich, M. P., Handbook of monoclonal antibodies: Applications in biology and medicine. Noges Publications, Park Ridge, N.J. (1985) ch. 22 and pp. 303-357.

Gregorek, H. et al. IgG subclass distribution of hepatitis B surface antigen antibodies induced in children with chronic hepatitis B infection after interferon-alpha therapy. J Infect Dis 181, 2059-2062 (2000).

Heijtink, R. A. et al. Characterization of a human monoclonal antibody obtained after immunization with plasma vaccine and a booster with recombinant-DNA hepatitis B vaccine. J Med Virol 66, 304-311 (2002).

Hyakumura, M. et al. Modification of Asparagine-Linked Glycan Density for the Design of Hepatitis B Virus Virus-Like Particles with Enhanced Immunogenicity. J Virol 89, 11312-11322 (2015).

Julithe, R., Abou-Jaoude, G. & Sureau, C. Modification of the hepatitis B virus envelope protein glycosylation pattern interferes with secretion of viral particles, infectivity, and susceptibility to neutralizing antibodies. J Virol 88, 9049-9059 (2014).

Kucinskaite-Kodze, I. et al. New broadly reactive neutralizing antibodies against hepatitis B virus surface antigen. Virus Res 211, 209-221 (2016).

Kim, S. H. & Park, S. Y. Selection and characterization of human antibodies against hepatitis B virus surface antigen (HBsAg) by phage-display. Hybrid Hybridomics 21, 385-392 (2002).

Li, D. et al. A potent human neutralizing antibody Fc-dependently reduces established HBV infections. Elife 6 (2017).

Lok, A. S., Zoulim, F., Dusheiko, G. & Ghany, M. G. Hepatitis B cure: From discovery to regulatory approval. Hepatology 66, 1296-1313 (2017).

Palanichamy, A. et al. Rituximab efficiently depletes increased CD20-expressing T cells in multiple sclerosis patients. J Immunol 193, 580-586 (2014).

Qiu X., et al., “Reversion of advanced Ebola virus disease in nonhuman primates with ZMapp” Nature 514 (7520): 47-53 (2014).

Rath, S. & Devey, M. E. IgG subclass composition of antibodies to HBsAg in circulating immune complexes from patients with hepatitis B virus infections. Clin Exp Immunol 72, 164-167 (1988).

Smith, T. W., Butler, V. P., Jr., Haber, E.: Cardiac glycoside-specific antibodies in the treatment of digitalis intoxication. In: Antibodies in Human Diagnosis and Therapy. Haber, E., Krause, R. M. (eds.) New York: Raven Press pp 365-389 (1977).

Trêpo, C., Chan, H. L. Y. & Lok, A. Hepatitis B virus infection. The Lancet 384, 2053-2063 (2014).

Tsai, T. H. et al. Study of IgG subclass profiles of anti-HBs in populations with different HBV infection status. Viral Immunol 19, 277-284 (2006).

Tsutsumi, Y. et al. Hepatitis B virus reactivation with a rituximab-containing regimen. World J Hepatol 7, 2344-2351 (2015).

Walsh, R. et al. Predicting HBsAg clearance in genotype A chronic hepatitis B using HBsAg epitope profiling: A biomarker for functional cure. Liver Int 39, 2066-2076 (2019).

Wang, W. et al. A human monoclonal antibody against small envelope protein of hepatitis B virus with potent neutralization effect. MAbs 8, 468-477 (2016).

WHO. Global Hepatitis Report 2017. (2017).

Zhang, T. Y. et al. Prolonged suppression of HBV in mice by a novel antibody that targets a unique epitope on hepatitis B surface antigen. Gut 65, 658-671 (2016).

1. An isolated neutralizing antibody or a fragment thereof that specifically binds to at least one conformational (non-linear) epitope of HBsAg and neutralizes infection with hepatitis B virus, wherein the antibody comprises variable light chain CDRL1, CDRL2 and CDRL3 amino acid sequences comprising the amino acid sequence set forth in SEQ ID NO: 3, SEQ ID NO: 4, and SEQ ID NO: 5, and variable heavy chain CDRH1, CDRH2, and CDRH3 amino acid sequences comprising the amino acid sequence set forth in SEQ ID NO:
6, SEQ ID NO: 7, and SEQ ID NO: 8.

2. The isolated neutralizing antibody or fragment thereof according to claim 1, wherein the antibody comprises at least one variable light chain and at least one variable heavy chain;
wherein the variable light chain amino acid sequence comprises the amino acid sequence set forth in SEQ ID NO: 1, and the variable heavy chain amino acid sequence comprises the amino acid sequence set forth in SEQ ID NO: 2; and/or
wherein the antibody has a constant region selected from the group comprising IgA, IgG1, IgG2, IgG3 and IgG4 subclass scaffolds; and/or
wherein the antibody is a fully-human antibody; and/or
wherein the hepatitis B virus genotype is selected from the group comprising genotype A (adw2), genotype B (adw), genotype C (adr) and genotype D (ayw).

3. A pharmaceutical composition comprising the isolated neutralizing antibody or fragment thereof defined in claim 1 and a pharmaceutically acceptable carrier.

4. (canceled)

5. (canceled)

6. A method of prophylaxis or treatment of HBV-infection and/or at least one HBV-linked disease, the method comprising administering to a subject an antibody or fragment thereof defined in claim 1.

7. The method according to claim 6, wherein the prophylaxis or treatment is for prophylaxis of perinatal HBV transmission, treatment of HBV positive mothers during the third trimester of pregnancy, prophylaxis of HBV recurrence in liver transplant recipients or HBV post-exposure prophylaxis in vaccine non-responders exposed to HBV, HBsAg-positive materials or treatment of chronically infected individuals.

8. An isolated nucleic acid molecule encoding:
(a) at least one variable light chain of the neutralizing antibody defined in claim 1; and
(b) at least one variable heavy chain of the neutralizing antibody defined in claim 1.

9. The isolated nucleic acid molecule according to claim 8, wherein the nucleic acid molecule encodes:
(a) at least one variable light chain of the neutralizing antibody, wherein the variable light chain comprises the amino acid sequence set forth in SEQ ID NO: 1; and
(b) at least one variable heavy chain of the neutralizing antibody, wherein the variable heavy chain comprises the amino acid sequence set forth in SEQ ID NO: 2.

10. The isolated nucleic acid molecule according to claim 9, wherein the at least one nucleic acid sequence in (a) has at least 80% sequence identity to SEQ ID NO: 9; and the at least one nucleic acid sequence in (b) has at least 80% sequence identity to SEQ ID NO: 10, due to redundancy of the genetic code.

11. An expression vector comprising at least one isolated nucleic acid molecule defined in claim 8.

12. A host cell comprising the expression vector of claim 11.

13. A kit comprising at least one neutralizing antibody or fragment thereof defined in claim 1.

14. A method of screening B cells for expression of virus-specific antibodies comprising:
a) coating the wells of an ELISA plate with a non-neutralizing virus-specific antibody;
b) adding the target virus to the wells for a period of time to allow binding of the virus to the virus-specific antibody;
c) adding media comprising antibodies, produced by activated memory B cells, to the wells for a period of time to allow binding of memory B cell virus-specific antibodies to the target virus;
d) adding labelled secondary antibodies to the wells to detect memory B cell virus-specific antibodies; and
e) identifying memory B cells that produced the virus-specific antibodies.

15. The method of claim 14, wherein the B cells are human B cells.

16. The method of claim 15, wherein the B cells have been clonally propagated; and/or
wherein the virus is HBV; and/or
wherein the labelled secondary antibody is an anti-human IgG antibody; and/or
wherein the memory B cells were isolated using a composition comprising antibodies directed to CD3, CD14, CD19, CD27, CD38, IgG and IgM; and/or
wherein the method further comprises isolating virus-specific antibodies from the identified memory B cells.


SEQUENCE LISTING (14 sequences)

SEQ ID NO: 1 - protein, 111 aa, synthetic construct
  REGION 1..111: note = HDAC006-11 light amino acid sequence
QLVLTQSPSASASLGASVKLTCTLSSGHGTYAIAWHQLRSEKGPRYLMKVNSDGSHIKGDGIPDRFSGSSSGAERYLIISSLQSEDEADYYCQTWGTGIVIFGGGTKLTVL

SEQ ID NO: 2 - protein, 135 aa, synthetic construct
  REGION 1..135: note = HDAC006-11 heavy chain amino acid sequence
QLQLQESGPGLVKPSETLSLTCTVSGGSMNINDYYWDWVRLSPGKGLEWIGAIYSSGTTYYNPSLKSRVTISVDMSKDQFSLRLSSVTAADTAVYYCVRDGPHRLYYGSGTYYNPRGGYYSYALDVWGQGTTVIV

SEQ ID NO: 3 - protein, 7 aa, synthetic construct
  REGION 1..7: note = HDAC006-11 light chain CDR1
SGHGTYA

SEQ ID NO: 4 - protein, 7 aa, synthetic construct
  REGION 1..7: note = HDAC006-11 light chain CDR2
VNSDGSH

SEQ ID NO: 5 - protein, 9 aa, synthetic construct
  REGION 1..9: note = HDAC006-11 light chain CDR3
QTWGTGIVI

SEQ ID NO: 6 - protein, 10 aa, synthetic construct
  REGION 1..10: note = HDAC006-11 heavy chain CDR1
GGSMNINDYY

SEQ ID NO: 7 - protein, 7 aa, synthetic construct
  REGION 1..7: note = HDAC006-11 heavy chain CDR2
IYSSGTT

SEQ ID NO: 8 - protein, 29 aa, synthetic construct
  REGION 1..29: note = HDAC006-11 heavy chain CDR3
VRDGPHRLYYGSGTYYNPRGGYYSYALDV

SEQ ID NO: 9 - DNA, 333 bp, synthetic construct
  misc_feature 1..333: note = HDAC006-11 light chain DNA sequence
  source 1..333: mol_type = other DNA
cagcttgtgttgactcaatcgccctctgcctctgcctccctgggagcctcggtcaagctcacctgcactctgagcagtgggcacggcacctacgccatcgcatggcatcaattgcggtcagagaagggccctcgatatttgatgaaagttaacagtgatggcagccacatcaagggggacgggatccctgatcgcttctcaggctccagttctggggctgagcgctacctcatcatctccagtttgcagtctgaagatgaggctgattattactgtcagacctggggcactggcattgtgatcttcggcggagggaccaagctgaccgtcctt

SEQ ID NO: 10 - DNA, 405 bp, synthetic construct
  misc_feature 1..405: note = HDAC006-11 heavy chain DNA sequence
  source 1..405: mol_type = other DNA
cagctgcaactgcaggagtcgggcccaggactggtgaagccttcggagaccctgtccctcacctgcactgtctctggtggctccatgaatattaatgattactattgggactgggtccgcctttccccaggaaagggactggagtggattggggctatctattctagtgggaccacctactataacccgtccctcaagagtcgagtcaccatatcagtagacatgtccaaggaccaattctccctgaggctgagctctgtgaccgccgcggacacggccgtttattattgtgtgagagatgggccccaccgtctctattacggttcggggacttattataacccgagagggggctactactcctacgctctggacgtctggggccaagggaccacggtcatcgtc

SEQ ID NO: 11 - protein, 226 aa, synthetic construct
  REGION 1..226: note = Amino acid sequence of HBsAg adw2
MENITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSMFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYI

SEQ ID NO: 12 - protein, 226 aa, synthetic construct
  REGION 1..226: note = Amino acid sequence of HBsAg adw
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWTSVRFSWLSLLVPFVQWFVGLSPTVWLSVIWMMWFWGPSLYNILSPFMPLLPIFFCLWVYI

SEQ ID NO: 13 - protein, 226 aa, synthetic construct
  REGION 1..226: note = Amino acid sequence of HBsAg adr
MENTASGFLGPLLVLQAGFFLLTRILTIPQSLDSWWTSLNFLGGAPTCPGQNSQSPTSNHSPTSCPPICPGYRWMCLRRFIIFLFILLLCLIFLLVLLDYHGMLPVCPLLPGTSTTSTGPCKTCTIPAQGTSMFPSCCCTKPSDGNCTCIPIPSSWAFARFLWEWASVRFSWLSLLVPFVQWFVGLSPTVWLSVIWMMWYWGPSLYNILSPFLPLLPIFFCLWVYI

SEQ ID NO: 14 - protein, 225 aa, synthetic construct
  REGION 1..225: note = Amino acid sequence of HBsAg ayw
MENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI